(19) United States
(12) Patent Application Publication
(10) Pub. No.: US 2016/0101167 A1
(43) Pub. Date: Apr. 14, 2016

(54) Composition of Organic Compounds

(71) Applicants: Peter Ulrich, Schopfheim (DE); Katja Baer, Schaffhausen (DE); Georges Imbert, Buschwiller (FR); Marie-Jose Hoellinger, Oberwil (CH); Marie-Emmanuelle Riviere, Saint Louis (FR); Ana Graf, Riehen (CH)

(72) Inventors: Peter Ulrich, Schopfheim (DE); Katja Baer, Schaffhausen (DE); Georges Imbert, Buschwiller (FR); Marie-Jose Hoellinger, Oberwil (CH); Marie-Emmanuelle Riviere, Saint Louis (FR); Ana Graf, Riehen (CH)

(73) Assignee: Novartis AG, Basel (CH)

(21) Appl. No.: 14/972,187

(22) Filed: Dec. 17, 2015

Related U.S. Application Data
(63) Continuation of application No. 14/751,492, filed on Jun. 26, 2015.
Application No. 14/751,492 is a continuation of application No. 14/456,562, filed on Aug. 11, 2014, now abandoned.
Application No. 14/456,562 is a continuation of application No. 13/578,017, filed on Aug. 9, 2012, now abandoned.
(60) Provisional application No. 61/361,697, filed on Jul. 6, 2010.

(30) Foreign Application Priority Data
Mar. 29, 2010 (EP) 10158273.2

(51) Int. Cl. A61K 39/00; A61K 45/06

(52) U.S. Cl. CPC A61K 39/0005; A61K 45/06; A61K 2039/55505; A61K 2039/545

(57) ABSTRACT

The present invention relates to compositions comprising a construct comprising the Aβ1-6 peptide and a pharmaceutically acceptable adjuvant, for the treatment of patients suffering from dementia, in particular dementia of the Alzheimer's type.

### TECHNICAL FIELD

The present invention relates to novel compositions and vaccines containing i) a construct comprising the Aβ1-6 peptide and ii) a pharmaceutically acceptable adjuvant (hereinafter Composition of the invention), and the use of such Compositions for the treatment of patients suffering from Alzheimer's disease (AD), in particular at an early stage of the disease.

### BACKGROUND ART

At least 15 million people are affected by Alzheimer's disease worldwide. This disease is characterized by a progressive impairment in patients' ability to function in daily life. Death occurs in most patients within 5 to 10 years of diagnosis.

Considerable evidence has been accumulated suggesting that the β-amyloid peptide—the major component of senile amyloid plaques—plays a causal role in AD. Successful disease-modifying therapy for AD is likely to include products that affect the deposition of β-amyloid in the brain. Aβ-specific antibodies, actively generated by the immune system or passively administered, consistently reduce plaque burden in different transgenic mouse models for Aβ-amyloidosis. A first clinical attempt to stimulate the immune system of AD patients to generate Aβ-antibody, however, had to be suspended due to unacceptable side effects (meningoencephalitis in 6% of treated patients, Orgogozo J M, Gilman S, Dartigues J F, Laurent B, Puel M, Kirby L C, Jouanny P, Dubois B, Eisner L, Flitman S, Michel B F, Boada M, Frank F, Hock C (2003) Subacute meningoencephalitis in a subset of patients with AD after Aβ42 immunization. Neurology; 61: 46-54). It was later concluded that in this trial Aβ-reactive autoimmune T cells were promoted, likely due to the activation of T_{H}1 lymphocytes. The T_{H}1 response was likely a result of the adjuvant used (QS-21) combined with T cell epitopes in the AN1792 (Lemere & Masliah, 2010, Nat. Rev. Neurol. 6(2):108-120). Thus, a careful choice of immunogen and adjuvant is needed to avoid such dangerous reactions while eliciting a useful immune response.

### DISCLOSURE OF THE INVENTION

Surprisingly, lesser adverse immune reactions and a lesser incidence of microhemorrhages are observed with constructs containing the Aβ1-6 peptide. In particular, no adverse immune reaction nor increased incidence of microhemorrhages, is observed with constructs consisting of a VLP chemically coupled to the Aβ1-6 peptide.

It was surprisingly found that constructs containing the Aβ1-6 peptide can advantageously be combined with an adjuvant, when being administered to humans suffering from dementia, Alzheimer's disease, dementia associated with Alzheimer's disease, or conditions related thereto.

Unexpectedly it has been found that administering an adjuvant together with a construct containing the Aβ1-6 peptide can be done without inducing a pro-inflammatory response although the antibody response to that construct increases. This is particularly important in aged patients.

As herein defined, “composition of the invention” refers to compositions comprising i) a construct comprising the Aβ1-6 peptide and ii) a pharmaceutically acceptable adjuvant. The composition of the invention may further comprise an acceptable pharmaceutical carrier.

According to the invention, the Aβ1-6 peptide is bound to a core particle having a structure with an inherent repetitive organization, for example a self-assembled virus-like particle (VLP). Such VLP may consist of capsid proteins of a RNA bacteriophage, for example capsid proteins of the RNA bacteriophage Qβ.

The fragment Aβ1-6 and construct containing such fragments as employed in the present invention are known as such. WO 04/016282 to Cytos and Novartis describes constructs comprising a VLP comprising recombinant proteins of a bacteriophage, such as Qβ, a linker and Aβ1-6, all together forming an ordered and repetitive antigen array.

The construct as employed in the present invention can be prepared, and purified as disclosed in WO 04/016282, especially in Example 13, the content thereof being incorporated by reference into the present patent application.

According to the invention, the VLP structure may be chemically coupled with a bivalent linker to the Aβ1-6 peptide. Such a bivalent linker may be as described in WO 04/016282, page 53, first paragraph, the content thereof being incorporated by reference.

In one embodiment the bivalent linker is a heterobifunctional cross-linker containing a functional group which can react with the virus-like particle or at least one virus-like particle subunit, for example the side-chain amino group of a lysine residue thereof. The bivalent linker may contain a further functional group able to react with the Aβ1-6 peptide or a cysteine residue fused to said Aβ1-6 peptide.

According to the invention, the heterobifunctional cross-linker may be selected from SMPH, Sulfo-MBS, Sulfo-EMCS, Sulfo-GMBS, Sulfo-SIAB, Sulfo-SMPB, Sulfo-SMCC, SVSB, SIA, for example SPDP or Sulfo-LC-SPDP.

In preferred embodiments of the invention, Aβ1-6 peptides suitable for generating the compositions of the invention are modified with an amino acid linker, e.g. an amino acid spacer, for binding to a VLP. Those Aβ1-6 peptides include, but are not limited to Aβ1-6 fused C-terminally to the spacer GGC. Amino acid linkers, e.g. amino acid spacers, suitable for fusion to the N-terminus of Aβ1-6 fragments include, but are not limited to the sequence CGG and CGHGNKS. Linkers suitable for fusion to the C-terminus of Aβ1-6 include but are not limited to the sequence GGC. In one embodiment, when a linker is fused to the C-terminus of the Aβ1-6 fragment, the C-terminal cysteine is amidated, which is indicated by the C-terminal “—CONH2”, and the N-terminus of the peptide is free, which is indicated by “NH2-”. In a specific embodiment, the amino acid linker, e.g. an amino acid spacer, containing a cysteine residue as second attachment site is fused to the C-terminus of the Aβ1-6 peptide.

In one embodiment, the construct comprising the Aβ1-6 peptide consists of a virus-like particle (VLP) of the RNA bacteriophage Qβ chemically coupled to said Aβ1-6 peptide with a bivalent linker, and wherein the Aβ1-6 peptide is modified with an amino acid spacer.

In another specific embodiment, the construct comprising the Aβ1-6 peptide consists of a virus-like particle (VLP) of the RNA bacteriophage Qβ, an Aβ1-6 peptide fused at its C-terminus to the spacer GGC, wherein the VLP is chemically coupled to said Aβ1-6 peptide with a bivalent linker; hereinabove defined as “Construct of the invention”.

In one aspect, the present invention provides for a vaccine composition comprising i) a construct comprising an Aβ1-6 peptide and ii) a pharmaceutically acceptable adjuvant, for example comprising the Construct of the invention and a pharmaceutically acceptable adjuvant.

The invention also provides therapeutic methods. Thus, the invention provides a composition of the invention or a vaccine of the invention for use in therapy. In another aspect, the present invention provides for a method of immunization comprising administering the composition of the invention, or vaccine of the invention, to an animal, e.g. a human.

### Adjuvants

As herein defined, the term “adjuvant” refers to an agent that when administered in conjunction (e.g. in combination) with the construct comprising the Aβ1-6 peptide of the invention, enhances the immune response to that construct. The adjuvant may increase the immune response by any of several mechanisms, such as lymphocyte recruitment, stimulation of B and/or T cells, and/or stimulation of macrophages.

According to the invention adjuvants can be, but are not limited to, organic, inorganic, oil-based adjuvants or virosomes.

Inorganic adjuvants include, but are not limited to mineral adjuvants, for example aluminium or calcium salts, such as aluminium phosphate, aluminium hydroxide (also referred to as Al(OH)₃ herein), potassium aluminium sulphate (also referred to as alum) and calcium phosphate. Such adjuvants may be used with or without other adjuvants, e.g. as mentioned below.

Organic adjuvants include, but are not limited to squalene.

Further examples of adjuvants according to the invention include, but are not limited to, MPL (Monophosphoryl Lipid A), AS03 (developed by GSK, Prepandrix), AS04 (developed by GSK; combination of MPL and aluminum hydroxide; Fendrix; Cervarix), QS21 (Saponin purified plant extract from the Soap bark tree (Quillaia saponaria) containing triterpene glucoside), AS01 (developed by GSK; liposomes; QS21 and MPL), AS02 (developed by GSK; QS21 and MPL), LT (heat labile enterotoxin from E. coli), CpG (oligonucleotides containing unmethylated CpG sequences), and MF59® (from Novartis). MF59 is a sub-micron oil-in-water emulsion of a squalene, polyoxyethylene sorbitan monooleate and sorbitan trioleate compounds.

Adjuvants particularly suitable for the invention are for example mineral adjuvants or adjuvants containing squalene, e.g. emulsion of squalene, e.g. MF59. In one embodiment, the composition of the invention comprises the Construct of the invention and either (i) MF59 or (ii) an aluminium salt (such as aluminium hydroxide).

The choice of adjuvant depends on the efficiency of adjuvant in promoting the immune response, the stability of the composition containing the adjuvant, e.g. the vaccine containing the adjuvant, the route of administration, the dosing regimen, the species to be vaccinated.

Two or more adjuvants can be combined. For example aluminium salts can be combined with MPL, QS21, and/or MF59.

According to the invention, about 5 to 600 μg of the construct comprising the Aβ1-6 peptide, e.g. the Construct of the invention, can be administered in human patients, for example about 5 to 550 μg, about 50 to 500 μg, about 100 to 500 μg, e.g. about 75 to 300 μg, e.g. about 50 to 150 μg, e.g. about 15 to 125 μs e.g. about 25 to 100 μg, e.g. about 50 μg, 75 μg, 100 μg, 150 μg, 200 μg, 300 μg, 400 μg or 450 μg. Thus, the composition of the invention may contain one of these amounts of the construct of the invention per dose. In one embodiment, the composition of the invention comprises about 150 μg or about 450 μg of the construct of the invention per dose.

In a specific embodiment, the Composition of the invention comprises about 10 to 600 μl/dose of adjuvant, e.g. about 50 to 500 μl/dose of adjuvant, e.g. about 100 to 500 μl/dose of adjuvant, e.g. about 100 to 300 μl/dose of adjuvant, e.g. about 150 to 300 μl/dose of adjuvant, e.g. about 125 to 250 μl/dose of adjuvant, e.g. about 125 μl/dose, or e.g. about 250 μl/dose or e.g. about 500 μl/dose of adjuvant. Such amounts are particularly suitable for MF59.

In one embodiment, the composition of the invention comprises i) about 100 to 300 μl/dose of MF59, e.g. about 125 μl/dose, about 250 μl/dose or about 500 μl/dose of MF59, and ii) about 150 μg of the construct comprising the Aβ1-6 peptide, for example 150 μg of the Construct of the invention. In one embodiment, the composition comprises (i) about 125 μl or about 250 μl MF59 and (ii) about 150 μg of the Construct of the invention per dose.

In another embodiment, the composition of the invention comprises i) about 100 to 500 μl/dose of MF59, e.g. about 125 μl/dose, 250 μl/dose, 450 μl/dose or 500 μl/dose of MF59, and ii) about 450 μg of the construct comprising the Aβ1-6 peptide, for example 450 μg of the Construct of the invention. In one embodiment, the composition comprises (i) about 125 μl or about 250 μl MF59 and (ii) about 450 μg of the Construct of the invention per dose.

In yet another embodiment, the composition of the invention comprises i) about 125 or 250 μl/dose of MF59, and ii) about 50 to 500 μg, about 100 to 500 μg, about 150 μg, e.g. about 200 μg of the construct comprising the Aβ1-6 peptide, for example 150 μg per dose of the Construct of the invention.

In another embodiment, the adjuvant is mixed with the construct comprising the Aβ1-6 peptide, for example with the Construct of the invention, in a ratio from about 0.5:1 (v/v) to about 4:1 (v/v), e.g. about 0.8:1 (v/v) to about 3.5:1 (v/v), e.g. about 1:1 (v/v) to about 2:1 (v/v); e.g. about 1:1 (v/v) ratio.

In another specific embodiment, the composition of the invention comprises about 10 to 900 μg/dose of adjuvant, e.g. about 50 to 850 μg/dose of adjuvant, e.g. about 100 to 800 μg/dose of adjuvant, e.g. about 120 to 600 μg/dose of adjuvant, e.g. about 100 to 550 μg/dose of adjuvant, e.g. about 150 to 450 μg/dose of adjuvant, e.g. about 50 μg/dose, about 100 μg/dose, about 150 μg/dose, or about 450 μg/dose of adjuvant. Such amounts are particularly suitable for aluminium salts, e.g. for Alum or aluminium hydroxide. The amount is based on weight of elemental aluminium in the case of aluminium hydroxide.

In one embodiment, the composition comprises (i) about 50 μg or about 150 μg aluminium hydroxide and (ii) 150 μg of the Construct of the invention per dose. In one embodiment, the composition comprises (i) about 150 μg or about 450 μg aluminium hydroxide and (ii) 450 μg of the Construct of the invention per dose.

In a further embodiment, the composition comprises (i) about 600 μg or about 850 μg aluminium hydroxide and (ii) about 450 μg of the Construct of the invention per dose.

If patient response is low, then an even a higher dosage form may be used. Thus in a further embodiment, the composition comprises (i) about 600 μg or about 850 μg aluminium hydroxide and (ii) about 600 μg of the Construct of the invention per dose.

When aluminium salts are used as adjuvants, suitable ratios of adjuvant to construct comprising the Aβ1-6 peptide include, but are not limited to, ⅓, ½, 1/1, 2/1, 3/1, 5/1 or 6/1 weight per weight based on elemental aluminum.

The adjuvant may be administered with the construct comprising the Aβ1-6 peptide as a single composition, or can be administered before, concurrent with or after administration of the construct comprising the Aβ1-6 peptide.

### Formulation and Administration

The Composition of the invention may be administered by various methods known in the art, e.g. by injection, infusion, inhalation, oral administration, or other suitable physical methods. The compositions may alternatively be administered intramuscularly, intravenously or subcutaneously. In a specific embodiment, the Composition of the invention is administered parenterally, e.g. intra muscularly or subcutaneously, for example intra muscularly.

Formulations containing the Composition of the invention include sterile aqueous, e.g. physiological saline; or non-aqueous solutions and suspensions. Examples of non-aqueous solvents are propylene glycol, polyethylene glycol, vegetable oils, such as olive oil, and injectable organic esters such as ethyl oleate. Carriers or occlusive dressings can be used to increase skin permeability and enhance antigen absorption.

For parental administration, the construct containing the Aβ1-6 peptide according to the invention can be administered as injectable dosages of a solution or suspension of said construct in a physiologically acceptable diluent with a pharmaceutically acceptable carrier which can be a liquid such as water, an oil, saline, glycerol or ethanol. Additional components may be included, such as wetting or emulsifying agents, surfactants, pH-buffering agents and the like. Other components may include petroleum, or oils of animal, vegetable or synthetic origin, for example peanut oil, soybean oil and mineral oil. Glycols such as propylene glycol and polyethylene glycol are particularly suitable carriers, e.g. for injectable solutions.

A suitable formulation for administering the Composition of the invention, e.g. for subcutaneous administration, is an aqueous solution containing Phosphate Buffer Saline (PBS) or another buffer. For example, the Composition of the invention contains between about 0.1 and 1 mg/mL of the Construct of the invention, e.g. between about 0.25 and 0.75 mg/mL of the Construct of the invention, e.g. between 0.4 and 0.6 mg/mL, e.g. 0.5 mg/mL of the Construct of the invention, and no further excipients. In one embodiment the invention provides an aqueous solution comprising Phosphate Buffer Saline (PBS) or another buffer and 1 mg/mL of the Construct of the invention.

The buffer may also contain L-histidine.

The Composition of the invention may further contain a bulking agent, e.g. sucrose. Hydrochloric acid may be added to adapt the pH.

The dosage form can be kept frozen or as lyophilisate until shortly before usage. Preferably, when in form of a lyophilisate the Composition of the invention contains a buffer (such as L-histidine) and a bulking agent, e.g. sucrose. Before administration, the lyophilisate is reconstituted with the appropriate volume of appropriate diluent, (for example water, or dextrose solution) in order to obtain the desired concentration of the construct comprising the Aβ1-6 peptide. Following addition of the diluent, the solution is gently mixed and left to rest until foam appears and the solution is clear and transparent. The reconstituted lyophilisate is then mixed with the appropriate adjuvant. Preferably the reconstituted lyophilisate mixed with adjuvant is not kept more than 4 hours at room temperature before administration.

Appropriate diluents include, but are not limited to, water, e.g. distilled water, physiological phosphate-buffered saline, Ringer's solutions, dextrose solution, Hank's solution. In one embodiment, the diluent may be the adjuvant itself. In one embodiment, the diluent is aluminium hydroxide solution.

The dosage form may be administered preferably by subcutaneous injection with a syringe to the warm-blooded animal, especially into the abdomen. In one embodiment, the composition (dosage form) is administered intramuscularly (i.e. is formulated for intramuscular administration). In one embodiment, the composition (dosage form) is injected into the upper arm.

For thawing of the dosage form, the dosage form can be kept at ambient temperature for between about 15 minutes and 45 minutes, e.g. 30 minutes. Preferably, before withdrawing drug substance, the vials are gently inverted several times for dispersion of potential sub-visual particles.

A suitable dosage form of the construct comprising the Aβ1-6 peptide according to the invention, e.g. the Construct of the invention, is a lyophilisate reconstituted in water for injection to obtain a concentration of the Construct of the invention of 1.0 mg/ml. This form is particularly suitable for administering the Construct of the invention in combination with a mineral adjuvant, e.g. with Aluminum hydroxide. Such a dosage form is particularly suitable for intramuscular administration of the Construct of the invention.

For administering the construct comprising the Aβ1-6 peptide according to the invention, e.g. the Construct of the invention, in combination with the adjuvant MF59 the dosage form is a lyophilisate reconstituted using adapted volumes of dextrose solution.

The Composition of the invention can be prepared as injectables, e.g. liquid solution or suspensions; or solid forms suitable for solution or suspension in liquid vehicles prior to injection.

### Therapeutic Methods

According to the invention, there is provided the use of the composition of the invention for the treatment and/or prevention of Alzheimer's disease (AD), especially at the early stage of the disease, or mild to moderate, or severe Alzheimer's disease (AD), or conditions related thereto. For example, there is provided the use of such compositions for the treatment or prevention of dementia, e.g. dementia associated with Alzheimer's disease and vascular dementia with amyloid angiopathy.

The present invention also provides the use of compositions of the invention for the treatment of patients with increased Aβ-level, including but not limited to patients with dementia associated with Parkinson's disease or Lewy Body dementia.

The present invention further relates to compositions of the invention for the prophylactic treatment of subjects at risk of developing AD, including but not limited to subjects with mild cognitive impairment, subjects with genotypes known to be associated with AD, such as ApoE4, subjects with Trisomy 21 and subjects with surrogate markers indicating risk for AD.

The term “treatment” as used herein relates in particular to a treatment aiming to halt the pathogenic processes that lead to disease progression and/or has symptomatic effects, or to attenuating the disease or the symptoms associated thereto.

The terms “dementia of the Alzheimer's type” (and “dementia associated with Alzheimer's disease”) as used herein relates in particular to a disease as defined according to the Diagnostic and Statistical Manual of Mental Disorders, 4th edition (DSM-IV) criteria.

The present invention also relates to a method of treatment of dementia, Alzheimer's disease, dementia associated with Alzheimer's disease or conditions related thereto in human patients comprising administering the composition of the invention to a patient in need thereof. The invention further provides immunization and vaccination methods, respectively, for preventing, treating and/or attenuating dementia, Alzheimer's disease, dementia associated with Alzheimer's disease or conditions related thereto in humans.

The frequency of injection can be varied depending on the patient response. For example the frequency of administration can varied by the attending physician depending on the patient's response and corresponding antibody titers. For example, a patient who is a low responder may require more frequent administration, while a patient who is a high responder may require less frequent administration in order to elicit and/or maintain the same antibody titer.

The frequency of injection can include, but is not limited to, 1 to 10 administrations per year, e.g. 2 to 8 per year, e.g. 6 administrations per year.

In one embodiment, the Composition of the invention is administered to human patients in need thereof about every 4 to 8 weeks, preferably about every 5 to 7 weeks, in particular about every 6 weeks. Such a dosing regimen may last about 12 to 16 weeks, e.g. to about 12 weeks. For example, the Composition of the invention is administered at 0, 6, 12 weeks. Furthermore, the delay between subsequent administrations of the Composition of the invention may be extended.

Thus, in one embodiment, the invention provides a dosing regimen of (a) two or more administrations at intervals of about 6 weeks, followed by (b) two or more administrations at intervals of about 12 weeks. In one embodiment, the invention provides a dosing regimen of (a) three administrations at intervals of about 6 weeks (e.g. at weeks 0, 6 and 12) followed by (b) two or more administrations (e.g. 3, 4, 5 or more) at intervals of about 12 weeks (e.g. at weeks 24, 36, 48 and 60).

Such a dosing regime is particularly suitable for treating patients suffering from dementia, Alzheimer's disease or dementia associated with Alzheimer's disease.

The usefulness of the Composition of the invention in the treatment of the above-mentioned disorders can be confirmed in suitable clinical studies, e.g. those described in the Examples.

Suitable clinical studies are in particular randomized, double-blind, placebo-controlled, parallel studies in Alzheimer's patients or open label studies.

### Combinations and Kits

The construct comprising the Aβ1-6 peptide and adjuvant may be packaged and supplied into the same container (e.g. vial or pre-filled syringe) or may be packaged in separate containers (e.g. vials) and mixed before use. Package, e.g. packaging, may include instructions to use, in particular when the Construct comprising the Aβ1-6 peptide and adjuvant are packaged separately, the package, e.g. packaging, typically includes instructions for mixing before use.

Thus, the invention provides a commercial package comprising: (a) a composition or vaccine of the invention, and (b) instructions for administration. The invention also provides a commercial package comprising: (a) the construct of the invention, (b) an adjuvant, and (c) instructions for use. In such a package, the construct may be lyophilised and the adjuvant may be used as a diluent. The kit may also optionally comprise a pharmaceutically acceptable diluent and/or an administration device (such as a syringe).

The invention also provides a commercial package comprising a) a construct comprising the Aβ1-6 peptide, e.g. the Construct of the invention, and b) adjuvant, together with (c) instructions for simultaneous, separate or sequential use thereof in the treatment or prevention of Alzheimer's disease or disorder associated thereto, in particular Alzheimer's disease.

In a further aspect, the present invention pertains to a combination comprising the Composition of the invention and at least one nootropic agent, preferably one cholinesterase-inhibitor, such as memantine.

The term “nootropic agent” as used herein includes, but is not limited to nootropic plant extracts, calcium antagonists, cholinesterase inhibitors, dihydroergotoxin, nicergoline, piracetame, purine derivates, pyritinol, vincamine and vinpocetine.

The term “nootropic plant extracts” as used herein includes, but is not limited to extracts from Ginkgo leafs. The term “calcium antagonists” as used herein includes, but is not limited to cinnarizine and nimodipine. The term “cholinesterase inhibitors” as used herein includes, but is not limited to donepezil hydrochloride, rivastigmine, memantine and galantamine hydrobromide. The term “purine derivates” as used herein includes, but is not limited to pentifyllin.

Extracts from Ginkgo leafs can be administered, e.g., in the form as marketed, e.g. under the trademark Ginkodilat™ according to the information provided by the package insert. Cinnarizine can be administered, e.g., in the form as marketed, e.g. under the trademark Cinnarizin Forte-Ratiopharm™. Nimodipine can be administered, e.g., in the form as marketed, e.g. under the trademark Nimotop™. Donepezil hydrochloride can be administered, e.g., in the form as marketed, e.g. under the trademark Aricept™. Rivastigmine can be prepared as disclosed in U.S. Pat. No. 5,602,176. It can be administered, e.g., in the form as marketed, e.g. under the trademark Exelon™. Galantamine hydrobromide can be administered, e.g., in the form as marketed, e.g. under the trademark Reminyl™ Dihydroergotoxin can be administered, e.g., in the form as marketed, e.g. under the trademark Hydergin™. Nicergoline can be administered, e.g., in the form as marketed, e.g. under the trademark Sermion™. Piracetam can be administered, e.g., in the form as marketed, e.g. under the trademark Cerebroforte™. Pentifyllin can be administered, e.g., in the form as marketed, e.g. under the trademark Cosaldon™. Pyritinol can be administered, e.g., in the form as marketed, e.g. under the trademark Encephabol™. Vinpocetin can be administered, e.g., in the form as marketed, e.g. under the trademark Cavinton™. Memantine can be administered, e.g., in the form as marketed, e.g. under the trademarks Axura™ or Namenda™.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

Hence, the present invention pertains also to a combination comprising a Composition of the invention and at least one nootropic agent selected from the group consisting of nootropic plant extracts, calcium antagonists, cholinesterase inhibitors, dihydroergotoxin, nicergoline, piracetame, purine derivates, pyritinol, vincamine and vinpocetine or memantine, in which the active ingredients are present in each case in free form or in the form of a pharmaceutically acceptable salt and optionally at least one pharmaceutically acceptable carrier, for simultaneous, separate or sequential use, especially for use in a method of treating dementia, Alzheimer's disease or disorder associated thereto.

Such a combination may be a combined preparation.

Other agents can be used in combination with the Composition of the invention, for example: antidepressants such as SSRIs, SNRIs, NRIs, antipsychotics such as risperidone, antidiabetic treatments such as insulin or metformin, antioxidative treatments such as selegiline, vitamin E, anti-inflammatory treatments such as NSAIDs, lipid-lowering agents such as statins, hormone substitution such as estrogens, amyloid lowering agents such as abeta secretase inhibitors, aggregation inhibitors such as beta-sheet blockers, chelators, immunomodulatory agents such as glatiramer acetate.

The term “a combined preparation”, as used herein defines especially a “kit of parts” in the sense that the active ingredients as defined above can be dosed independently or by use of different fixed combinations with distinguished amounts of the ingredients, i.e., simultaneously or at different time points. The parts of the kit can then, e.g., be administered simultaneously or chronologically staggered, that is at different time points and with equal or different time intervals for any part of the kit of parts. Preferably, the time intervals are chosen such that the effect on the treated disease in the combined use of the parts is larger than the effect which would be obtained by use of only any one of the active ingredients.

Hence, the present invention also provides:
a combination as disclosed herein for use in therapy.
the use of a combination as disclosed herein for the preparation of a medicament for the prevention and/or treatment of Alzheimer's disease or disorder associated thereto, such as dementia; in particular Alzheimer's disease, e.g. at the early stage of the disease.
a commercial package comprising a combination as disclosed herein together with instructions for simultaneous, separate or sequential use thereof in the prevention and/or treatment of Alzheimer's disease or disorder associated thereto such as dementia, in particular Alzheimer's disease, e.g. at the early stage of the disease.

In one embodiment, the invention provides (a) a composition of the invention, in combination with (b) a combination partner. In one embodiment of the invention, the combination partner (b) is a cholinesterase inhibitor, especially rivastigmine, or memantine.

If the combination partners are administered as separate dosing forms, a dosage and mode of administration can be applied as provided in the package inserts. In particular, the following dosages of the combination partners (b) can be administered to the patient: Cinnarizine may be administered to a patient in a total daily dosage of between about 75 to about 150 mg.

Nimodipine may be administered to a patient in a total daily dosage of between about 60 to about 120 mg.

Donepezil hydrochloride may be administered to a patient in a total daily dosage of between about 5 mg and 10 mg.

Rivastigmine may be administered to a patient in a total daily dosage of between about 2 and about 20 mg, e.g. about 4 and about 18 mg, e.g. about 6 and about 12 mg.

Galantamine may be administered to a patient in a total daily dosage of between about 12 and 24 mg, e.g. 12 mg twice daily.

Dihydroergotoxin may be administered in the form of its methanesulfonate to a patient in a total daily dosage of between about 4 mg and 10 mg, e.g. about 8 mg.

Nicergoline may be administered in the form of its tartrate by intramuscular injection to a patient in a total daily dosage of between about 4 mg and 8 mg.

Piracetam may be administered to a patient in a total daily dosage of between about 1200 and 5000 mg, e.g. 4800 mg/day.

Pentifyllin may be administered to a patient in a total daily dosage of between about 400 and 800 mg.

Pyritinol may be administered in the form of its hydrochloride to a patient in a total daily dosage of about 600 mg.

Vinpocetin may be administered to a patient in a total daily dosage of between about 10 and 15 mg.

Memantine may be administered to a patient in the form of memantine hydrochloride in a total daily dosage of about 20 mg.

The invention also provides a container containing the composition, vaccine or combination of the invention. The container may be made of glass or a plastic. The container may have a sterile access port. Suitable containers included within the scope of the invention include bottles, vials, syringes and test tubes.

### General

The term “comprising” means “including” as well as “consisting” e.g. a composition “comprising” X may consist exclusively of X or may include something additional e.g. X+Y.

The term “about” in relation to a numerical value x means, for example, x+10%.

The invention having been fully described, it is further illustrated by the following examples and claims, which are illustrative and are not meant to be further limiting. Those skilled in the art will recognize or be able to ascertain using no more than routine experimentation, numerous equivalents to the specific procedures described herein. Such equivalents are within the scope of the present invention and claims.

### EXAMPLES

### Example 1

### Intramuscular Injections of a Composition Containing the Construct of the Invention and Aluminium Hydroxide (Al(OH)₃) or MF59 to Rabbits

Six groups of rabbits consisting of 9 females/group are treated by intramuscular injection in the hindlimb on Day 1 (upper part of the thigh muscle, right hindlimb), Day 14 (upper part of the thigh muscle, left hindlimb) and Day 28 (lower part of the thigh muscle, right hindlimb). Group 1, 2 and 3 are treated with 150 μg Construct of the invention mixed with 0.050 mg Al(OH)₃ (Group 1), 0.150 mg Al(OH)₃ (Group 2) or 0.450 mg Al(OH)₃ (Group 3). Groups 4, 5 and 6 are treated with 150 μg Construct of the invention mixed with 0.125 mL MF59 (Group 4), 0.25 mL MF59 (Group 5) or 0.5 mL MF59 (Group 6). The volumes of MF59 comprised 5, 10.0 or 20.0 mg Squalene, respectively, which is the active principle in MF59. The animals are necropsied 14 days after the last administration (Day 42).

The following parameters are evaluated: mortality/viability (twice daily), clinical signs (daily) including skin reactions at the intramuscular sites of injection (approximately 24 and 48 hours after dosing), body weights (weekly), food consumption (twice weekly), hematology and clinical biochemistry (once during pre-treatment and on Day 42), macroscopic examination at termination, organ weights, and histopathology of the injection sites. Blood samples for serological analysis are taken (once during pre-treatment and on Days 20, 26, 34, 39 and 42). In addition, samples of plasma (once during pre-treatment and on Day 42) and cerebrospinal fluid (at necropsy on Day 42) are collected.

No mortality, nor any toxicologically relevant changes in clinical signs, skin observations, body weight (gain), (relative) food consumption, haematological and clinical biochemistry parameters, and necropsy findings has been noted.

An immunogenic response is noted in all treated animals. For all groups, maximal mean concentrations of anti-Abeta and anti-Q-beta (response to carrier) IgG has been reached on study Day 34, i.e. six days after the third injection, and decreased on Days 39 and 42. The anti-Abeta IgG production in Groups 1 to 5, but not including Group 6, exhibits a marked inter-animal variability. The anti-Qbeta immune response shows less inter-animal variability than the anti-Abeta immune response. The animals that either do not respond or weakly respond to Abeta, conversely respond well to Qbeta. For both adjuvants tested (i.e. Al(OH)₃ or MF59) the maximum mean anti-Abeta IgG concentrations increases between the first and second dose.

For the rabbits treated with the Construct of the invention plus Aluminium hydroxide, the response consists mainly of a dose dependent increase in incidence and severity of the macrophage response with some lymphocytic inflammation. The response resolves over time, returning to background levels following the earliest injection (Day 1 injection).

For the rabbits treated with the Construct of the invention plus MF59 (Groups 4, 5 and 6), there is an increased incidence in inflammatory reactions in a dose (but not time) dependent manner but no increase in the severity of the observations is noted.

This study shows that in female Albino New Zealand White rabbits, three intramuscular injections of the Construct of the invention in combination with Aluminium hydroxide or MF59 were well tolerated. In all groups, maximal mean concentrations of anti-Abeta and anti-Q-beta IgG concentrations are reached on Day 34 of the treatment phase, i.e. six days after the third injection, and decrease on Days 39 and 42. In comparison to the Construct of the invention/Al(OH)₃-treated groups treated with the Construct of the invention and Al(OH)₃, co-administration of the Construct of the invention and MF59 results in a slightly higher immunogenic response for both anti-Abeta and anti-Qbeta IgG. The high dose of either adjuvant does not provide any advantage over the respective intermediate dose.

### Example 2

### Intramuscular Injections of a Composition Containing the Construct of the Invention and Aluminium Hydroxide (Al(OH)₃) or MF59 to Monkeys

The objective of the study is immunization with the Construct of the invention alone or in combination with aluminium hydroxide or MF59, to old female cynomolgus monkey (Macaca fascicularis) over at least 26 weeks. The animals are dosed on study days: 1, 15, 43, and 140.

The following investigations respectively samplings are performed: mortality, clinical observations (incl. post dose observations of the injection sites), body weights, neurological assessment, neurobehavioral observations, serology (antibody Abeta and Qbeta titer determination), PBMC collection for T-cell stimulation, proteomics and metabolomic (results—proteonics and metabolonic—reported separately), hematology, clinical chemistry and urine analysis, weighing and histological processing of selected organs/tissues, microscopic observations (including IHC and silver staining of brain regions for amyloid plaque determination and CSF analysis).

The following dose levels are selected:

TABLE 1
Selected dose levels

| | | | | Dose | | | Nec- |
|---|---|---|---|---|---|---|---|
| | | | Dose | level | Animals/ | | ropsy |
| | | Group | volume | (μg/ | group | | after |
| Group | adju- | descrip- | (mL/per | injec- | | Fe- | 28 |
| number | vant | tion | animal) | tion) | Males | males | weeks |
| 1 | s.c | Low 1 | 0.150 | 150 | None | 5 | 5 |
| 2 | s.c | High 1 | 0.400 | 400 | None | 5 | 5 |
| 3 | aluminium | High 1 | 0.550* | 400 | None | 5 | 5 |
| | hydroxide, | | | | | | |
| | s.c | | | | | | |
| 4 | MF59 i.m. | High 1 | 0.800 | 400 | None | 5 | 5 |

*rounded from 0.548 mL/per animal;
s.c.: subcutaneously;
i.m.: intramuscular

No test item-related deaths or test item-related findings are observed in any of the evaluated parameters during the conduct of the study. There is no macroscopic or histopathological evidence of target organ toxicity due to test substance administration.

Findings at necropsy are consistent with the expected spectrum of background pathology in cynomolgus monkeys. There are no unusual macroscopic findings suggestive of target organ toxicity.

Histopathological findings are generally consistent with the expected background pathology in aged female cynomolgus monkeys. Subcutaneous or intramuscular administration of Construct of the invention with and without the adjuvants aluminium hydroxide and MF59 is well tolerated at dose levels of 150 or 400 μg/day on days 1, 15, 43, and 140 of the study to female geriatric cynomolgus monkeys and gives no indication of systemic test item toxicity. No treatment or dose level-related effects are observed during the conduct of the study.

### Example 3

### 26-Week Subcutaneous and Intramuscular Injection in Cynomolgus Monkey

The study is conducted with the Construct of the invention in combination with adjuvants, and the application of seven clinical immunizations via the subcutaneous (s.c.) and intramuscular (i.m.) route for using Al(OH)₃ and the i.m. route for MF59.

The following investigations are performed: concentration verification, clinical observations, body weights, neurological examinations, neurobehavioral observations, ophthalmic examinations, electrocardiography, blood pressure, serology (analysis of anti Abeta/Qbeta specific IgGs), PBMC collection for T cell stimulation and ELISPOT analysis, A beta analysis, hematology, clinical chemistry, urine analysis, immunoglobulin determinations, CSF sampling, organ weights, macroscopic examination at necropsy, and histopathology. The following dose levels are selected:

TABLE 2
Selected dose levels

| | | | Injection | Dose | | |
|---|---|---|---|---|---|---|
| | | | volume | level | | |
| Group | adju- | Group | (mL/per | (μg/in- | Animals/group | |
| number | vant | description | animal) | jection) | Males | Females |
| 1 | s.c | Al(OH)₃ | 0.77 | 0 | 3 | 3 |
| 2 | i.m. | Al(OH)₃ | 0.77 | 0 | 3 | 3 |
| 3 | i.m. | MF59 | 1.2 | 0 | 3 | 3 |
| 4 | s.c. | Construct/ | 0.77 | 600 | 4 | 4 |
| | | Al(OH)₃ | | | | |
| 5 | i.m. | Construct/ | 0.77 | 600 | 4 | 4 |
| | | Al(OH)₃ | | | | |
| 6 | i.m. | Construct/ | 1.2 | 600 | 4 | 4 |
| | | MF59 | | | | |

The results of this study can be summarized as follows: Animal 24965F of group 5 is killed moribund on day 91 of the study, due to diarrhea and severe body weight loss. Hematological evaluation on day 91 shows a slightly reduced hematocrit value and slightly increased monocytes. Clinical chemistry shows moderately increased blood urea and unbalanced electrolytes as well as reduced total protein, albumin, and globulin. Key findings of this markedly emaciated animal at necropsy are abnormal semifluid contents of the large intestine associated with red mucosal discolouration in cecum and colon.

Histopathologically, slight (colon) and moderate (cecum) crypt microabscesses are identified as related to the semifluid contents of the large intestine. This is accompanied by moderate villous atrophy of the ileum. A number of other findings indicate the impaired condition of the animal with reduced food intake over a prolonged duration. Since these findings are observed in only one animal, they are considered to be incidental and are not related to treatment with the test item/aluminium hydroxide combination.

After subcutaneous injection, severe changes including swelling and erythema are seen at the injection sites in animals that are administered the Construct of the invention (600 μg/injection) combined with Al(OH)₃ (group 4). Similar findings, but less severe are seen in the control group (group 1) where only Al(OH)₃ is given. Swelling and erythema are also seen after intramuscular administration of Construct of the invention (600 μg/injection) combined with Al(OH)₃ (group 5), but also with less severity.

No other findings are observed during the in-life phase that could be related to the Construct of the invention or the adjuvants MF59 or Al(OH)₃ or any combinations.

All animals demonstrate Abeta and Qbeta IgG antibody responses after treatment with the Construct of the invention. No Abeta antibody titers are observed in control groups, with one exception (group 3, animal 24886, day 152: 9.3 units). Qbeta antibody titers in the control groups are in most cases below the limit of quantification. However, in 12 samples out of 252, values above LLOQ are measured (6 in group 1; 1 in group 2; 5 in group 3). In groups 4 and 5, Qbeta titers are measured in 4 out of 16 predose samples (2 in group 4; 2 in group 5). The range of these Qbeta values (from control groups 1 to 3 or from predose samples (groups 4 to 6) is very low (range: 1.0 to 3.4 units) compared to the values observed after treatment with the Construct of the invention in groups 4, 5, and 6 (all animals had at least one post-dose Qbeta titer value superior or equal to 841.7 units). In general a strong increase in antibody titers is observed after the third injection and the following injections: Abeta and Qbeta IgG profiles are very similar in all groups treated with the Construct of the invention. Addition of aluminium hydroxide induces slightly higher Abeta titers than addition of MF59. The effect is more pronounced when Qbeta titers are considered. Mode of administration of the Construct of the invention+aluminium hydroxide (s.c. or i.m.) has very little impact on Abeta and Qbeta immune response. While the ELISPOT assay detects a induced expansion of Qbeta specific T cells, the data show the absence of Abeta specific T cell expansion.

Changes seen histopathologically at the injection sites of groups 1, 2, 4, and 5 (receiving Al(OH)₃ or Construct of the invention/Al(OH)₃ combination) included histiocytosis and subacute inflammation of varying severity. Colliquative necrosis is seen in the centers of larger histiocyte accumulations in groups 4 and 5 (Construct of the invention/Al(OH)₃ combination s.c. and i.m., respectively). Clusters of histiocytes of similar appearance occur in the draining lymph nodes (axillary in group 4, inguinal in group 5) in single animals of groups 4 and 5. At the injection sites of group 6 (Construct of the invention/MF59) minimal to moderate subacute inflammation in 3 males and minimal and slight subacute inflammation in three females are observed.

In conclusion, subcutaneous and intramuscular administration of the test item (Construct of the invention) in combination with Al(OH)₃ leads to local swelling and erythema. Histopathologically, histiocytosis and subacute inflammation as well as colliquative necrosis and clusters of histiocytes in the draining lymph nodes are observed.

Administration of the adjuvant Al(OH)₃ also leads to swelling, erythema, histiocytosis and subacute inflammation at the injection sites, but at less severity. Therefore, it can be concluded that the test item contributes to these findings if it is administered in combination with Al(OH)₃.

Administration of the test item in combination with MF59 was associated with only subacute inflammation which was not seen in the corresponding control group (group 3).

### Example 4

### A 90-Week, Randomized, Double-Blind, Placebo-Controlled Study in Patients with Alzheimer's Disease with Repeated Intramuscular Injections of the Construct of the Invention

Patients are below 85 years of age (inclusive), with mild AD as confirmed by a Mini-Mental State Examination (MMSE) score of 20 to 26 (both inclusive). Patients are untreated or on stable dose of cholinesterase inhibitor or memantine over the last 4 weeks prior to clinical assessments. They are randomized to receive repeated intra-muscular injections of the adjuvanted Construct of the invention or placebo. A first pool of patients receive repeated intra-muscular injections of 150 μg Construct of the invention plus either 150 μg Aluminium hydroxide, 50 μg aluminium hydroxide, 250 μl MF59, or 125 μl MF59. A second pool of patients receive repeated intra-muscular injections of 450 μg Construct of the invention plus either 150 μg aluminium hydroxide, 450 μg aluminium hydroxide, 125 μl MF59, or 250 μl MF59. A third pool of patients receive repeated intra-muscular injections of placebo containing either 150 μg aluminium hydroxide, 450 μg aluminium hydroxide, 125 μl MF59, or 250 μl MF59.

The dosings are given at weeks 0, 6, 12 and then at weeks 24, 36, 48 and 60.

Aluminium hydroxide is manufactured by diluting the bulk suspension (15 g/L Al(OH)₃) with a NaCl solution. The final composition of the suspension is 2.7 mg/ml Al(OH)₃, 9 mg/ml NaCl pH 5.9 (pH 5.5-7.5). The homogeneous suspension is filled into 2 ml vials, sealed with rubber stoppers, autoclaved for sterility and stored at 2° C.-8° C.

In case of MF59, the bulk material is aseptically filled in 3 ml vials, sealed with stoppers and stored at 2° C.-8° C. protected from light.

The Construct of the invention is mixed with the adjuvant prior to administration.

Safety assessments include general physical examinations, neurological examinations, 12-lead electrocardiograms (ECGs), vital signs, standard clinical laboratory evaluations (hematology, blood chemistry, urine analysis), special immunological laboratory evaluations in blood and cerebrospinal fluid (CSF), cerebral magnetic resonance imagings (MRIs), as well as adverse event and serious adverse event monitoring.

Aβ-antibody response is measured by determination of the Aβ-antibody titer (IgG and IgM) in serum and CSF using ELISA methods. The ex vivo Aβ-antibody binding properties in serum and CSF is explored by immunological methods on human and β-amyloid precursor protein (APP) transgenic mouse brain tissue. The VLP-antibody titer response in serum is measured to investigate the immune response to the carrier compound in relation to the immune response to Aβ.

Exploratory pharmacodynamic assessments include the following assessments: 1) determination of disease related markers in CSF (Aβ peptides and its isoforms, tau protein and its isoforms, phospho-tau) and plasma (Aβ peptides and isoforms); 2) volumetric MRIs, and 3) Alzheimers disease Assessment Scale (ADAS)-cognitive subscale, mini-mental state examination (MMSE), clinical dementia rating (CDR) and Alzheimer's Disease Cooperative Study—Activities of Daily Living (ADCS-ADL).

Responders are defined as those patients who show a significant increase of Aβ-specific antibody titers above baseline. Aβ-specific antibody titers are defined as titers above lower limit of quantification (LLOQ) in a validated enzyme-linked immunosorbent assay (ELISA) assay detecting specific antibodies relative to a standard serum as calibrator.

1. A composition comprising i) a construct comprising the Aβ1-6 peptide bound to a virus-like particle and ii) a pharmaceutically acceptable adjuvant, wherein said adjuvant is Al(OH)₃ in an amount of about 50 μg to about 850 μg Al(OH)₃ per dose of said construct.

2. The composition according to claim 1, comprising between 5 to 600 μg of the construct comprising the Aβ1-6 peptide.

3. The composition according to claim 1, comprising 150 μg or 450 μg of the construct comprising the Aβ1-6 peptide.

4. The composition according to claim 1, wherein the construct comprising the Aβ1-6 peptide is in aqueous solution.

5. The composition according to claim 1, wherein the construct comprising the Aβ1-6 peptide consists of a virus-like particle (VLP) structure chemically coupled to the Aβ1-6 peptide.

6. The composition according to claim 5, wherein the VLP is from the RNA bacteriophage Qβ, the Aβ1-6 peptide is fused at its C-terminus to the spacer GGC, and wherein the VLP is chemically coupled to said Aβ1-6 peptide with a bivalent linker.

7. The composition according to claim 1, wherein the composition comprises about 50 μg, about 150 μg, about 450 μg, about 600 μg or about 850 μg Al(OH)₃ per dose of said construct.

8. The composition according to claim 1, comprising, per dose of said construct:
(i) about 150 μg of the construct comprising the Aβ1-6 peptide and about 150 μg Al(OH)₃;
(ii) about 450 μg of the construct comprising the Aβ1-6 peptide and about 150 μg Al(OH)₃;
(iii) about 450 μg of the construct comprising the Aβ1-6 peptide and about 450 μg Al(OH)₃;
(iv) about 450 μg of the construct comprising the Aβ1-6 peptide and about 600 μg Al(OH)₃;
(v) about 450 μg of the construct comprising the Aβ1-6 peptide and about 800 μg Al(OH)₃;
(vi) about 600 μg of the construct comprising the Aβ1-6 peptide and about 600 μg Al(OH)₃; or
(vii) about 600 μg of the construct comprising the Aβ1-6 peptide and about 800 μg Al(OH)₃.

9. The combination comprising a composition according to claim 1 and at least one agent selected from the list of nootropic agents, memantine, antidepressant agents, antipsychotic agents, antidiabetic agents, antioxidative agents, anti-inflammatory agents, lipid-lowering agents, hormone substitution agents, amyloid lowering agents, aggregation inhibitors, chelators, and immunomodulatory agents.

10. The method of treating or preventing dementia, Alzheimer's disease, dementia associated with Alzheimer's, or other disorders related to increased Aβ levels in a subject in need thereof, comprising administering a composition according to claim 1 or the combination according to claim 9 to said subject.

11. The method according to claim 10, wherein the composition or combination is administered at intervals of about 6 to about 12 weeks.

12. The method according to claim 10, wherein the composition or combination is administered twice or more at intervals of about 6 weeks and then twice or more at intervals of about 12 weeks.

13. A commercial package comprising: (a) the construct according to claim 1, (b) Al(OH)₃, and (c) instructions for use.

14. The composition according to claim 1, wherein the composition is in a container selected from the group consisting of a bottle, a vial, a syringe or test tube.
